(19) United States
(12) Patent Application Publication
(10) Pub. No.: US 2002/0127536 A1
(43) Pub. Date: Sep. 12, 2002

(54) Mitochondrial assay

(76) Inventor: June R. Aprille, Andover, MA (US)

Correspondence Address: C. Hunter Baker, M.D., Ph.D., Choate, Hall & Stewart, Exchange Place, 53 State Street, Boston, MA, 02109, US

(21) Appl. No.: 09/758,554

(22) Filed: Jan. 11, 2001

(51) Int. Cl. C12Q 1/00; A61K 31/122; A61K 31/19

(52) U.S. Cl. 435/4; 514/682; 514/557

(57) ABSTRACT

The disclosed invention provides a method and system of assaying chemical compounds to determine their effect on mitochondrial cellular respiration. Many neuroleptics have been implicated as interfering with mitochondrial function, and these effects may lead to long term side effects such as extrapyramidal side effects and tardive dyskinesia. In order to develop better pharmaceutical agents with fewer side effects, the mitochondrial assay as disclosed herein allows one to screen many drug candidates quickly for their effect on mitochondrial function and predict long term side effects.

### BACKGROUND OF THE INVENTION

Schizophrenia is a devastating psychiatric condition which occurs in about 1% of the population. Although the causes of schizophrenia are not completely known, symptoms of the disease include paranoia, delusions, auditory and visual hallucinations, and disturbances of emotion. Schizophrenia is characterized as a mental state of impaired reality testing, disordered behavior, and thought disturbances. It tends to have its onset in the second or third decade of life and is generally a lifelong condition.

One of the more popular theories for understanding schizophrenia is the dopamine hypothesis. This hypothesis postulates that overactivity of dopaminergic neurotransmitter pathways in the brain leads to schizophrenia. Many of the therapeutics used currently in the treatment of schizophrenia block the neurotransmitter dopamine. These therapeutics are commonly known as antipsychotics or neuroleptics. Many of these dopamine receptor antagonists have especially strong binding to D₂ receptors in the subcortical and mesolimbic tracts where many of the symptoms of schizophrenia are thought to arise. Examples of neuroleptics include haloperidol, chlorpromazine, clozapine, risperidone, olanzapine, quetiapine, and thioridazine.

Although the neuroleptics do not provide a cure for schizophrenia, they can induce the remission of psychotic symptoms and help to prevent the recurrence of symptoms. These medications also have the drawback of causing many side effects including extrapyramidal symptoms (EPS), Parkinsonian side effects (e.g., abnormal gait, masked faces, and difficulty in initiating movement), akathisia, gynecomastia, galactorrhea, neuroleptic malignant syndrome, and tardive dyskinesia (TD).

In contrast to many of the other side effects of neuroleptics, tardive dyskinesia (TD) usually occurs after cumulative exposure to medications with first onset at about six months. Women (especially post-menopausal women), some ethnic groups, and the elderly appear at the highest risk for developing TD. Also unlike other side effects, TD is irreversible, and the best treatment for TD is prevention; patients therefore should be maintained on the lowest doses of neuroleptics possible without the recurrence of psychosis. One sign of TD is oro-buccal-lingual motion (as if the patient were chewing gum).

Tardive dyskinesia is one of the most problematic extrapyramidal movement disorders produced by the chronic administration of neuroleptic drugs due to its high prevalence and frequently irreversible course (Tarsy et al., “Tardive Dyskinesia” Annu. Rev. Med. 35:605-623, 1984; incorporated herein by reference). Despite the promise of new atypical or novel antipsychotics with their low, if not negligible, incidence of extrapyramidal symptoms (EPS), conventional antipsychotics remain widely used clinically, and therefore a substantial number of patients remain at risk for TD. Moreover, when atypical antipsychotics are used at higher doses and for longer periods of time, they may be found to produce TD and other EPS. Although considerable effort has been directed towards elucidating the molecular mechanism of TD, its cause remains unknown.

Although the pathophysiology of TD remains poorly understood, numerous theories have been proposed including dopamine receptor supersensitivity (Burt et al., “Antipsychotic drugs: chronic treatment elevates dopamine receptor binding in brain” Science 1966:326-328, 1977; incorporated herein by reference), catecholamine hyperactivity (Kaufmann et al., “Noradrenergic and neuroradiological abnormalities in tardive dyskinesia” Biol. Psychiat. 21:799-812, 1986; Saito et al., “Neurochemical findings in the cerebral spinal fluid of schizophrenic patients with tardive dyskinesia and neuroleptic-induced Parkinsonism” Jpn. J. Psychiat. Neurol. 40:189-194, 1986; each of which is incorporated by reference), and GABA hyperactivity (Gale, “Chronic blockade of dopamine receptors by antischizophrenic drugs enhances GABA binding in substantia nigra” Nature 283:569-570, 1980; incorporated herein by reference). Of these, the dopaminergic receptor theory has received the most attention, in part due to the prominent role dopamine appears to play in schizophrenia and the dopaminergic receptor antagonism of typical neuroleptics. Dopamine receptor supersensitivity, where small changes in exogenous dopamine receptors lead to an exaggerated dopamine-mediated response, is thought to occur with chronic dopamine receptor antagonism. However, there are difficulties with this theory, stemming from studies that failed to demonstrate a significant increase in D₂ receptor binding in postmortem brain when comparing dyskinetic versus non-dyskinetic controls (Fields et al., “Neurochemical basis for the absence of overt stereotyped behaviors in rats with up-regulated striatal D₂ dopamine receptors” Clin. Neuropharmacol. 14:199-208, 1991; Kornhuber et al., “³H spiperone binding sites in postmortem brains from schizophrenic patients: relationship to neuroleptic drug treatment, abnormal movements, and positive symptoms” J. Neurol. Transm. 75:1-10, 1989; each of which is incorporated herein by reference). Also, only a poor correlation between neuroleptic-induced dopamine supersensitivity in animal models and human TD has been demonstrated (Andersson et al., “Striatal binding of ¹¹C-NMSP studied with positron emission tomography in patients with persistent tardive dyskinesia: no evidence for altered D₂ receptor binding” J. Neural Trans. Gen. Sect. 79:215-226, 1990; Knable et al., “Quantitative autoradiography of striatal dopamine D₁, D₂ and re-uptake sites in rats with vacuous chewing movements” Brain Res. 646:217-222, 1994; each of which is incorporated herein by reference).

An alternative hypothesis, which is gaining support in explaining the pathogenesis of TD, involves neuroleptic-induced impairment of striatal energy metabolism. Evidence for this phenomenon was first provided by Mitchell et al. (Mitchell et al., “Regional changes in 2-deoxyglucose uptake associated with neuroleptic-induced tardive dyskinesia in the cebus monkey” Mov. Disord. 7:32-37, 1992; incorporated herein by reference), who showed that neuroleptic treatment sufficient to produce TD leads to regional changes in glucose utilization. This is particularly relevant to TD since it has been known for many years that neuroleptics have inhibitory effects on mitochondrial respiratory enzyme activities (Maurer et al., “Inhibition of complex I by neuroleptics in normal human brain cortex parallels the extrapyramidal toxicity of neuroleptics” Molec. Cell Biochem. 174:255-259, 1998; Prince et al., “Neuroleptic-induced mitochondrial enzyme alterations in rat brain” J. Pharmacol. Exptl. Therapeut. 280:261-267, 1977; Gallager et al., “The effect of phenothiazine on the metabolism of rat liver mitochondria” Biochem. Pharmacol. 10:369-372, 1965; each of which is incorporated herein by reference). More recently, Burkhardt et al. have shown that typical neuroleptics inhibit mitochondrial respiratory chain activity at concentrations 100-fold lower than an atypical compound such as clozapine (Burkhardt et al., “Neuroleptic medications inhibit complex I of the electron transport chain” Ann. Neurol. 33:512-517, 1993; incorporated herein by reference). These findings have led to the suggestion that typical antipsychotics, which exert their action predominately at D₂ receptors, affect oxidative metabolism in neurons of the striatal-nigral pathway, resulting in cell dysfunction and/or death thus causing perturbed motor control characteristic of TD. Also, Roberts et al. demonstrated that neuroleptics induce specific morphological alterations of mitochondria in rat striatum (Roberts et al, “Ultrastructural correlates of haloperidol-induced oral dyskinesias in rat striatum” Synapse 20:234-243, 1995; each of which is incorporated herein by reference). In addition, Prince et al. reported a significant reduction of mitochondrial respiratory complex I activity in the striatum and nucleus accumbens of rats treated with typical antipsychotics (Prince et al., “Neuroleptic-induced mitochondrial enzyme alteration in the rat brain” J. Pharmacol. Exp. Ther. 280:243-261, 1997; incorporated herein by reference). Similarly, Anderssen and Jorgensen found that a mitochondrial-specific toxin induced vacuous chewing movements, the rodent equivalent of TD (Anderssen et al., “The mitochondrial toxin 2-nitropropionic acid induces vacuous chewing movements in rats. Implications for tardive dyskinesia?” Psychopharmacol. 119:474-476, 1995; incorporated herein by reference). Lastly, Goff et al. demonstrated impaired brain energy metabolism in patients with neuroleptic-induced TD by measuring the level of Krebs cycle intermediates in the CSF (Goff et al., “Tardive dyskinesia and substrates of energy metabolism in CSF” Am. J. Psychiatr. 152:1730-1736, 1995; incorporated herein by reference).

Similar mechanisms have been offered to explain the pathology of neurodegenerative diseases (Beal et al., “Do defects in mitochondrial energy metabolism underlie the pathology of neurodegenerative diseases?” Trends Neurosci. 16:125-131, 1993; incorporated herein by reference). For example, in both Alzheimer's disease (AD) and Huntington's disease (HD), primary defects in brain oxidative metabolism are thought to contribute to excitotoxic cell injury and the subsequent abnormal movements characteristic of these disorders. Deficits in mitochondrial electron transport have been demonstrated in both AD and HD (Kish et al., “Reduced activity of mitochondrial cytochrome c oxidase in postmortem samples of Alzheimer's disease brain” J. Neurochem. 59:776-779, 1992; Brennan et al., “Regional mitochondrial activity in Huntington's disease brain” J. Neurochem. 44:1948-1950, 1985; each of which is incorporated herein by reference).

Neuroleptic-induced Parkinsonism and idiopathic Parkinson's disease (PD) are quite similar clinically, and neuroleptics certainly have been shown to exacerbate parkinsonism. Considerable evidence now suggests that deficits in mitochondrial respiratory chain activity may be an important factor in the development of Parkinson's disease (Schapira, “Evidence for mitochondrial dysfunction in Parkinson's Disease-a critical appraisal” Mov. Disord. 9:125-138, 1991; Parker et al., “Abnormalities of the electron transport chain in idiopathic Parkinson's Disease” Ann. Neurol 26:719-723, 1989; each of which is incorporated herein by reference). Assays of platelets, muscle, and brain from PD patients have shown specific decreases in mitochondrial complex I activity (Shoffner et al., “Mitochondrial oxidative phosphorylation defects in PD” Ann. Neurol. 30:332-339, 1991; incorporated herein by reference). The 1-methyl-4-phenyl-1,2,3,6-tetrahydropyridine (MPTP) model of PD involves the production of the pyridinium ion MPP⁺ by glial cell MAO-B, which is taken up by neuronal cell dopamine transporters and concentrated in mitochondria. MPP⁺ is a potent and specific inhibitor of mitochondrial respiratory complex I (Ransay et al., “The energy-driven uptake of N-methyl-4-phenylpyridine by brain mitochondria mediates the neurotoxicity of MPTP” Life Sci. 39:581 -588, 1986; incorporated herein by reference). Interestingly, MPTP is structurally similar to haloperidol, which is converted intracellularly to HPP⁺, a pyridinium species similar to MPP⁺ (Subramanyam et al., “Identification of a potentially neurotoxic pyridinium metabolite of haloperidol in rats” Biochem. Biophys. Res. Comm. 166:238-244, 1990; Subramanyam et al., “Studies on the in vitro conversion of haloperidol to a potentially neurotoxic pyridinium metabolite” Chem. Res. Toxicol. 4:123-128, 1991; Niklas et al., “Inhibition of NADH-linked oxidation to brain mitochondria by 1-methyl-4-phenylpyridinium, a metabolite of the neurotoxin 1-methyl-4-phenyl-1,2,3,6-tetrahydropyridine” Life Sci. 38:2503-2508, 1985; each of which is incorporated herein by reference). Rolema et al. have recently shown that HPP⁺ is also a potent inhibitor of mitochondrial complex I (Rollema et al., “Neurotoxicity of a pyridinium metabolite derived from haloperidol: in vivo microdialysis and in vitro mitochondrial studies” J. Pharmacol. Exp. Ther. 268:380-387, 1994; incorporated herein by reference).

A method for screening for side effects of chemical compounds in the various stages of their development as pharmaceutical agents, including long term side effects such as those resulting from the disruption of mitochondrial bioenergetic function, would be very useful in the pharmaceutical industry.

### SUMMARY OF THE INVENTION

The present invention provides methods and systems for determining the effect of a test chemical compound on mitochondrial bioenergetic function and the likelihood of related side effects when the compound is used as a drug. In this invention, the test compound is added to intact mitochondria or intact mitochondrial membranes to determine its effect on the integrated bioenergetic functions. After the test compound is added to the intact mitochondria, a characteristic of mitochondrial oxidative phosphorylation is measured. Preferred characteristics include, but are not limited to, for example, oxygen uptake, rate and energetic efficiency of ATP production, rate of ADP utilization, NADH consumption, FADH₂ consumption, respiratory rates with various reduced substrates (linked by NADH, FADH₂, electron-transferring flavoprotein (ETF), or others), production of reactive oxygen species, permeability changes, substrate transport, membrane potential, ion transport, etc. Specific mitochondrial bioenergetic functions to be measured include 1) uncoupling between electron transport and ATP synthesis; 2) inhibition of electron transport, potentially at specific respiratory complexes; 3) inhibition of ATP synthetase; and/or 4) volume changes.

Any observed effects on respiration in the intact mitochondria can optionally be followed up by direct enzyme assays to pinpoint any site-specific effects. For example, the enzymatic activities of NADH-cyt c reductase, succinate-cyt c reductase, cytochrome c oxidase, ATP synthetase, and/or succinate dehydrogenase can be assayed. Site-specific inhibition of electron transport can also be confirmed by assaying oxidized-reduced difference spectra. Interaction of the test compound with the permeability transition pore can be tested separately as well.

In another aspect of the invention, an observed effect on mitochondrial function is correlated with a negative reaction when the test compound is administered to an individual to provide a method of predicting long-term side effects. For example, in the case of neuroleptics, the data from the effects of typical and atypical drugs on mitochondrial function can be correlated with the known incidence and patterns of EPS effects such as tardive dyskinesia for each drug. This will allow one to predict potential adverse effects caused by the long term use of new antipsychotics. Application of these data and correlations to other drug families will allow one of skill in this art to develop all types of drugs with no EPS effects and no risk of the recipient developing movement disorders such as TD in the future.

The present invention provides a system for evaluating test compounds based on their effects on mitochondria and by inference on cellular bioenergetic function. The system for evaluating test chemical compounds comprises a test compound, intact mitochondria, and a means of measuring a characteristic of mitochondrial respiration.

This mitochondrial assay will allow one to predict potential adverse effects caused by the use of typical as well as atypical antipsychotics. Lastly, the ability to rapidly screen test compounds through the mitochondrial assay will be important in refining drug design to obtain clinically efficacious compounds with fewer unwanted side effects.

### DRAWINGS

FIG. 1 shows the various protein complexes involved in mitochondrial oxidative phosphorylation in the mitochondrial membranes. The figure shows how electrons enter the pathway from three types of oxidizable substrates: those that are NADH-linked, entering through Complex I; those that enter through flavins in the ETF complex; and those that are FADH₂-linked, entering through Complex II. Electrons from all three sources are collected by CoQ and passed successively through Complex III, cytochrome c, Complex IV, and finally to oxygen. The energy released by these electron transfers is used to pump H⁺ in Complexes I, III, and IV, which creates a H⁺ concentration gradient across the inner membrane. This gradient is the force that drives ATP synthesis through the ATP synthetase enzyme (far right).

FIG. 2 shows representative data from isolated rat liver mitochondria assayed polarographically to detect drug inhibition of electron transport. A known uncoupler (2,4-dinitrophenol) was added in the presence of an oxidizable substrate to elicit maximal electron transport rates, measured as the maximal rate of oxygen consumption; a slower rate in the presence of the drug indicates inhibition somewhere in the electron transport chain. The assay is performed with different substrates that donate electrons at either respiratory complex I (glutamate+malate) or at complex II (succinate) or through electron-transferring flavoprotein (ETF) (not shown) to gain information about site-specific inhibition.

FIG. 3 shows representative data from isolated rat liver mitochondria assayed polarographically to detect uncoupling action of test compounds. Normally electron transport proceeds at a slow basal rate in the presence of an oxidizable substrate unless the H⁺ gradient is dissipated by a need for ATP synthesis. However, if the H⁺ gradient is dissipated by the addition of a chemical that alters membrane permeability to H⁺, respiration will be stimulated unproductively, that is, without concomitant ATP synthesis, so that energy is wasted. To determine whether a drug might have such an uncoupling effect, the drug is added to mitochondria respiring in the basal state with an oxidizable substrate present. If respiration (oxygen consumption) is stimulated, uncoupling is inferred. A known uncoupler like 2,4-dinitrophenol will stimulate basal respiration 6-10 fold.

FIG. 4 shows representative data from isolated rat liver mitochondria assayed polarographically to detect drug inhibition of ATP synthetase. When coupling is intact, basal respiration will be stimulated by the addition of ADP to invoke ATP synthesis, as electron transport is stepped up to replace the H⁺ gradient that is used by the ATP synthetase enzyme. If the drug inhibits this coupled respiration rate, the inhibition could be due either to inhibition of the ATP synthetase itself or to inhibition of the electron transport rate that supports the ATP synthesis. To detect which is inhibited, the drug is also tested for inhibition of maximal electron transport rates in the presence of a known uncoupler. Inhibition of the coupled, but not the uncoupled respiration rate pinpoints the inhibition to the ATP synthetase or to one of the supporting transport processes such as the phosphate or ADP/ATP transporters.

### DETAILED DESCRIPTION OF PREFERRED EMBODIMENTS

The present invention uses intact mitochondria or mitochondrial membranes to screen compounds for their effect(s) on mitochondrial respiration and correlates the effect on respiration with potential clinical side effects such as extrapyramidal symptoms and tardive dyskinesia. This system and method are particularly useful in the development of new drugs. This screening method will predict acute effects and may also predict side effects which may not appear until a recipient has used a drug for months, years, or even decades.

General Principles ofMitochondrial Oxidative Phosphorylation

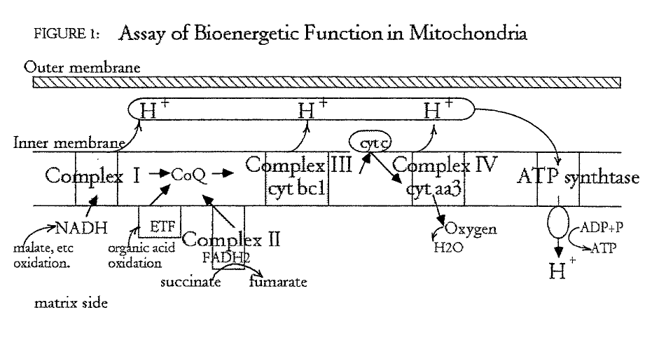

The driving force for bioenergetic function is electron transport (cellular respiration). High energy electrons from NADH and FADH₂ and other reduced coenzyme intermediates which have been produced during the metabolism of fuels such as carbohydrates and fats (e.g., glycolysis, organic acid oxidation, and the citric acid cycle) are used in the reduction of O₂ to H₂O. As the high energy electrons are passed through the redox reactions of the electron transport chain located in the lipid bilayer of the inner mitochondrial membrane, a proton gradient is created as H⁺ is transported from the inside to the outside of the mitochondrial inner membrane (FIG. 1).

The membrane is not freely permeable to H⁺, so the proton gradient becomes a source of potential free energy that can be coupled to drive other reactions. The coupling between the dissipation of the proton gradient and the synthesis of ATP from ADP and phosphate is known as oxidative phosphorylation. ATP synthesis is driven by the controlled movement of H⁺ down its concentration gradient. As the protons move through the ATP synthetase enzyme which spans the membrane, the free energy that is released is coupled to the formation of the phosphodiester bond of ATP and release of the ATP product. Mitochondria are vulnerable to drug interference in several well-established ways including 1) inhibition of electron transport at specific sites, 2) uncoupling of the proton concentration gradient and ATP synthesis, 3) specific and non-specific inhibition of transport proteins and ATP synthetase, and 4) cellular consequences of perturbed bioenergetic function-energy status and apoptosis.

Test Compounds

Compounds used in the present invention may be any chemical compound. Chemical compounds include organic compounds, inorganic compounds, organometallic compounds, salts, and metals. In a preferred embodiment, the chemical compounds are organic compounds with pharmaceutical activity. In another embodiment of the invention, the chemical compound is a clinically used drug. In a particularly preferred embodiment, the drug is a neuroleptic. Not only neuroleptics but other drugs including peptide drugs, protein drugs, polynucleotides, oligonucleotides, antibiotics, anti-viral agents, steroidal agents, anti-inflammatory agents, anti-neoplastic agents, antigens, vaccines, antibodies, decongestants, antihypertensives, sedatives, birth control agents, progestational agents, anti-cholinergics, analgesics, anti-depressants, anti-psychotics, β-adrenergic blocking agents, diuretics, cardiovascular active agents, non-steroidal anti-inflammatory agents, nutritional agents, etc. may be assayed using the methods and systems of the present invention. In other preferred embodiments, the chemical compounds assayed include environmental toxins, polymers, natural products, small molecules, and naturally occurring substances.

In the case of neuroleptics, the new atypical antipsychotics are marketed as having a low to no potential for EPS effects in general and TD in particular; however, similar enthusiasm followed the introduction of the high potency neuroleptics, only to find out several years later the development of TD. Only as these newer compounds are used more frequently, at higher doses, and for longer periods of time will one be able to assess fully their potential for adverse reactions. Lastly, patients will continue to receive typical neuroleptics for a variety of clinical reasons and therefore undesirable side effects such as TD will continue to appear in medicine.

Mitochondria

Mitochondria may be isolated from any source, fungi or animals. In a preferred embodiment the mitochondria used in the present invention are isolated from an animal source, and more preferably from a mammalian source. One particular embodiment uses mitochondria from rat which are isolated using techniques known in the art. The mitochondria may be isolated from any type of tissue, cell, or cell line. Preferred cell types include myocytes, neurons, and hepatocytes. Preparations of these mitochondria including intact mitochondria isolated from a cell or tissue, mitochondria with the outer membrane disrupted, mitochondria with both the outer and inner membranes disrupted, and isolated functional membranes of mitochondria are used to assay chemical compounds for their effects on bioenergetic function.

In a preferred embodiment, when the compound to be tested will be used as a drug administered to a patient, the mitochondria are isolated from cells of the same species as the patient being treated. In a particularly preferred embodiment, the mitochondria are isolated from cells from a relative or from the patient. The cells may be obtained by a biopsy of the patient and grown in cell culture if need be. The closer the mitochondria are to the patient's mitochondria the better the model for predicting side effects when the drug is administered to the patient.

In another preferred embodiment of the present invention, when a large quantity of mitchondria are needed, the mitchondria may be obtained from fungi (e.g., yeast), from animal tissues, or from cells grown in tissue culture.

Assaying

To assay for the effect of the test compound on the mitochondria, the test compound is contacted with the mitochondria preparation. Preferably, the compounds to be tested are added over a concentration range of 1 to 100,000 μM to establish a dose-response relationship for effects. More preferably, the concentration of the compound is 1-500 μM. Any characteristic of mitochondrial respiration may be measured to assess the compound's effect on the mitochondria. These characteristics include oxygen consumption with various oxidizable substrates, spectral characteristics of a protein (e.g., cytochromes) in the mitochondrial respiratory chain, rate and energetic efficiency of ATP production, rate and energetic efficiency of ADP utilization, respiratory rates with various reduced substrates (linked by NADH, FADH₂, ETF, or others), production of reactive oxygen species, membrane permeability changes, substrate transport, membrane potential, ion transport, and concentration of an electron donor (e.g., FADH₂, NADH). Those of ordinary skill in this art will recognize that any of a variety of different tests could be performed. To exemplify, described below are four particularly interesting, but non-limiting examples of assays of mitochondrial function.

Inhibition of electron transport.

This first test answers the questions of whether the compound under study inhibits electron transport, and if so, whether inhibition involves specific respiratory enzyme complexes in the electron transport chain. A known uncoupler of oxidative phosphorylation (e.g., 2,4-dinitrophenol) is added to the preparation to elicit maximal electron transport rates, measured as the rate of oxygen consumption. A slower rate in the presence of the drug indicates inhibition somewhere along the electron transport chain. The test is performed with different substrates that donate electrons at either respiratory complex I (glutamate and malate) or at complex II (succinate) or via electron-transferring flavoprotein (ETF) to gain information about site-specific inhibition.

Pilot studies have shown that some of the antipsychotic drugs are site-specific inhibitors of mitochondrial respiration (FIG. 1). Site-specific inhibition at respiratory complexes III or IV inhibits the transport of electrons from all sources, since these complexes are the final common pathway to the reduction of molecular oxygen. Not surprisingly, human mitochondrial diseases that affect complex II and IV have early onset and are very often fatal. In contrast, genetic disorders of complex I are more slowly debilitating, presumably because there are alternative pathways to CoQ (Aprille, “Mitochondrial cytopathies and mitochondrial DNA mutations” Current Opinion in Pediatrics 3:1045-1054, 1991; incorporated herein by reference). Increased production of reactive oxygen species is one direct result of partial inhibition at complex I, II, and III and leads to oxidative stress (Glinn et al., “Initiation of lipid peroxidation in submitochondrial particles: Effect of respiratory inhibitors” Archiv. Biochem. Biophys. 290:57-65, 1991; Cadenas et al., “Enhancement of hydrogen peroxide formation by protophores and ionophores in antimycin-supplemented mitochondria” Biochem. J. 188:1-37, 1980; each of which is incorporated herein by reference) that is a precursor for mitochondria-induced apoptosis (for an excellent review, see Green et al., “Mitochondria and apoptosis [Review]” Science 281:1309-1312, 1998; incorporated herein by reference). Well-known site-specific inhibitors include rotenone and MPP⁺, which inhibit complex I; antimycin-a, myxothiazol, and ubiquinol analogues of CoQ, which inhibit complex III; and carbon monoxide, cyanide, and hydrogen sulfide, which inhibit complex IV (Rollema et al, “Neurotoxicity of a pyridinium metabolite derived from haloperidol: in vivo microdialysis and in vitro mitochondrial studies” J. Pharmacol. Exp. Ther. 268:380-387, 1994; Lash et al., eds., Methods in Toxicology, Vol. 2: Mitochondrial Dysfunction, Academic Press, San Diego, 1993; each of which is incorporated herein by reference).

The role of mitochondria in apoptosis includes an effect of second messengers and reactive oxygen species on the “permeability transition pore” that spans both mitochondrial membranes (Green et al., “Mitochondria and apoptosis [Review]” Science 281:1309-1312, 1998; Zamami et al., “Mitochondrial control of Nuclear Apoptosis” J. Exptl. Med. 183:1533-1544, 1996; each of which is incorporated herein by reference). The pore complex includes a protein that specifically binds benzodiazepines and whose function is ill-defined, and these receptors are induced by steroid hormones and are somehow necessary for the transport of cholesterol into mitochondrial sites of steroid synthesis (Gavish et al., “The endocrine system and mitochondrial benzodiazepine receptors” Molec. Cell. Endocrinol. 88:1-13, 1992; Krueger et al, “Mitochondrial benzodiazepine receptors and the regulation of steroid biosynthesis” Ann. Rev. Pharmacol. Toxicol. 32:211-237, 1992; Whitehouse, “Benzodiazepines and steroidogenesis” Endocrinol. 134:1-3, 1992; each of which is incorporated herein by reference). Certain of the antipsychotic medications, particularly those in the diazepine family, may affect apoptosis through interaction with this intracellular receptor, with potential to regulate the permeability transition pore.

Non-specific inhibition of electron transport can also occur. The respiratory enzyme complexes are multimeric proteins that react with their substrates by diffusional collisions in the membrane lipid bilayer. Lipophilic chemical compounds can inhibit by disrupting protein-lipid interactions that are important to the functional integrity of the respiratory complexes. Another type of inhibitor includes compounds that can produce reactive oxygen species that can then oxidize lipids, nucleic acids, and proteins (Ara et al., “Mechanisms of mitochondrial photosensitization by the cationic dye, N,N-Bis(2-ethyl-1,3-dioxylene)kryptocyanine (EDKC): preferential inactivation of complex I in the electron transport chain” Cancer Res. 47:6580-6585, 1987; Modica-Napolitano et al., “Mitochondrial toxicity of cationic photosensitizers for photochemotherapy” Cancer Res. 50:7876-7881, 1990; each of which is incorporated herein by reference). Complex I is the most susceptible to these kinds of non-specific inhibitors because it consists of at least 25 subunits and is functionally the most labile of the electron transport enzymes (Rouslin, “Identification of mitochondrial dysfunction at coupling site I: loss of activity of NADH-unbiquinone oxidoreductase during myocardial ischemia” Chapter 26, 207-218; incorporated herein by reference).

Uncoupling.

The second test determines whether the drug uncouples electron transport from ATP synthesis. Normally, in these preparations, electron transport will proceed at a slow basal rate in the presence of an oxidizable substrate, unless the H⁺ gradient is dissipated by a need for ATP synthesis. However, if the H⁺ gradient is dissipated by the addition of a drug that alters membrane permeability to H⁺, respiration will be stimulated unproductively (i.e., without concomitant ATP synthesis) so that energy is wasted. To determine whether a drug might have such an uncoupling effect, the drug is added to mitochondria respiring in the basal rate. If respiration (e.g., oxygen consumption) is stimulated, uncoupling is inferred.

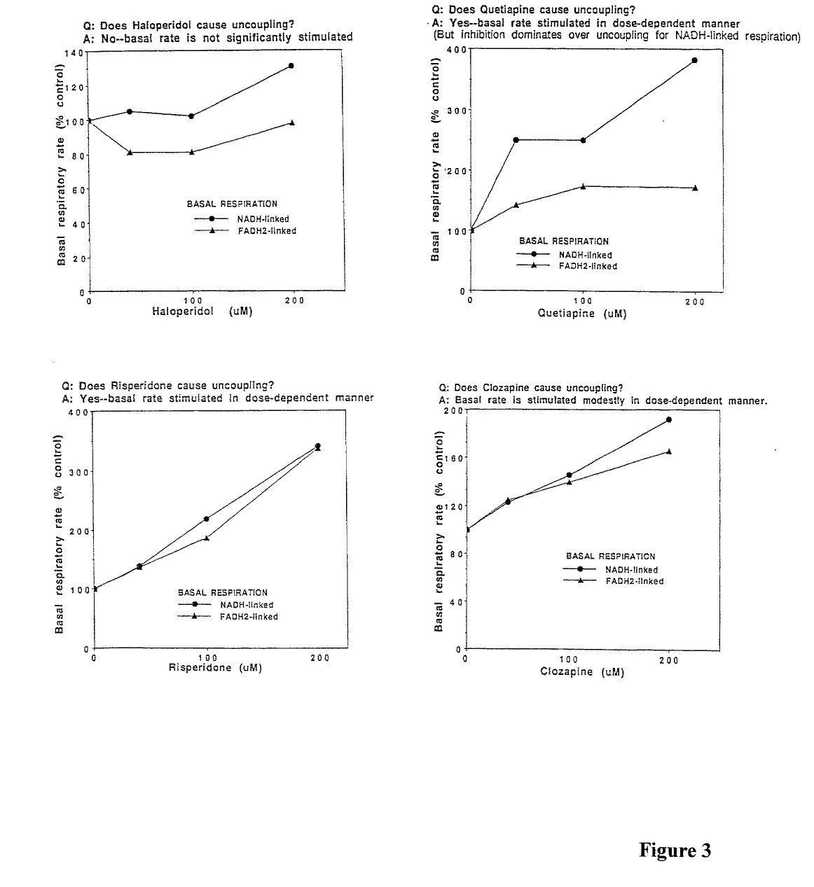

Uncoupling means the wasting of the proton concentration gradient without coupling to any productive transport function or to ATP synthesis. Uncouplers can be permeable proton ionophores (e.g., 2,4-dinitrophenol) or lipophilic compounds that disrupt membrane permeability in a non-specific way. Uncouplers stimulate electron transport, which works in vain to restore the H⁺ gradient. Complete uncoupling is incompatible with life; however, partial uncoupling will increase the metabolic rate, which is another cause of increased production of reactive species (Rand, “Thermal habit, metabolic rate and the evolution of mitochondrial DNA” Trends Ecol. Evol. 9:125-131, 1994; Loft et al., “Oxidative DNA damage correlated with oxygen consumption in humans” FASEB Journal 8:534-537, 1994; each of which is incorporated herein by reference). Partial uncoupling also will alter membrane potential, affect steady state ATP/ADP and AND(P)H/AND(P) ratios, and membrane potential dependent calcium homeostasis. In studies so far (FIG. 3), none of the antipsychotics tested are potent uncouplers at pharmacologic concentrations; however, their metabolites could act as uncouplers (i. e., if the metabolite is both lipophilic and a weak acid).

Specific or non-specific inhibition of transport proteins and ATP synthetase.

The third test examines whether the drug inhibits ATP synthetase. When coupling is intact, basal respiration will be stimulated by the addition of ADP to invoke ATP synthesis, as electron transport is stepped up to replace the H⁺ gradient that is used by the ATP synthetase enzyme. If the drug inhibits this coupled respiration rate, the inhibition could be due either to inhibition of the ATP synthetase itself or to inhibition of the electron transport rate that supports the ATP synthesis. To detect the difference, the drug is also tested for inhibition of maximal electron transport rates in the presence of a known uncoupler, as in the first test described supra. Inhibition of the coupled but not the uncoupled respiration rate pinpoints the inhibition to the ATP synthetase or to one of the supporting transport processes such as phosphate or ADP/ATP transporters.

Each of the unique proteins that are important for the coordinated function of oxidative phosphorylation are potential targets for inhibition. These targets include the ATP synthetase itself (Modica-Napolitano et al., “Basis for the selective cytotoxicity of Rhodamine 123” Cancer Res. 47:4361-4365, 1987; incorporated herein by reference) and supporting transport reactions such as ADP/ATP translocase, substrate transport, and phosphate transport (Aprille, “Mechanism and regulation of the mitochondrial ATP-Mg/Pᵢ carrier” J. Bioenerg. Biomembranes 25:473-481, 1993; incorporated herein by reference). Inhibition at any of these sites will compromise rates of ATP synthesis. Of the drugs tested, only clozapine so far is a candidate for inhibition of ATP synthetase.

Cellular consequences of perturbed bioenergetic function: energy status and apoptosis.

Inhibition of electron transport can compromise the rate of ATP synthesis in cells resulting in diminished oxidative capacity and energy status. In neuronal cells, a functional inability to maintain membrane potentials may be one consequence, possibly leading to excitotoxic cell death. Chronic depolarization could also lead to cell death through loss of regulation of voltage-regulated channels, such as NMDA, which then results in massive calcium influx.

The role of mitochondria in apoptosis has been the focus of recent intensive research (for excellent review, see Green et al., “Mitochondria and apoptosis [Review]” Science 281:1309-1312, 1998; incorporated herein by reference). Inhibition of electron transport, particularly at respiratory complex I, produces excess oxygen radicals that trigger opening of the mitochondrial transition pores. These pores release cytochrome c from the mitochondria into the cytosol; in vertebrates, cytosolic cytochrome c is a signal that triggers the sequence of molecular events that regulates apoptosis. Thus, an interesting hypothesis to explain extrapyramidal side effects such as TD involves inhibition of electron transport enzymes with consequent apoptosis of cells that take up neuroleptic drugs.

Pharmaceutical Compositions

Bioenergetic defects induced by neuroleptic treatment or any other type of chemotherapeutic treatment may suggest specific therapeutic interventions. First, an attempt to bypass or ameliorate the bioenergetic lesion could be considered by administration of compounds that are coenzymes of the respiratory chain enzymes such as coenzyme Q (Przyrembel, “Coenzyme Q10: a potential mediator of excitoxic cell damage of the mitochondrial electron transport chain” J. Inherit. Metab. Dis. 10:129-146, 1987; incorporated herein by reference), which could bridge a defect in the electron transport chain. Another possibility might be to provide alternative substrates such as hydroxybutyrate, which bypasses inhibition at complex I. Pharmaceutical composition may then be designed using the data from the mitochondrial assay to minimize the side effects of the pharmaceutical agent. A therapeutically effective amount of a drug may be combined with, for example, a vitamin, coenzyme Q, or alternative substrate (e.g., hydroxybutyrate) to yield a pharmaceutical composition with reduced side effects when compared to the drug alone. The pharmaceutical compositions of the present invention may be administered by any known method including, for example, intravenous, intramuscular, subcutaneous, intrasternal, intraosseous, and parenteral administration.

These and other aspects of the present invention will be further appreciated upon consideration of the following Examples, which are intended to illustrate certain particular embodiments of the invention but are not intended to limit its scope, as defined by the claims.

### Definitions

Animal refers to human as well as non-human animals. Non-human animals include, for example, mammals, birds, reptiles, amphibians, and fish. Preferably, the non-human animal is a mammal (e.g, a rodent, a mouse, a rat, a rabbit, a monkey, a dog, a cat, or a pig). An animal includes transgenic animals.

Intact refers to a mitochondrial bioenergetic system which is functional and/or capable of coupled oxidative phosphorylation. Intact mitochondria include mitochondria isolated from the cell of origin, mitochondria with the outer membrane disrupted but the inner membrane intact, and isolated portions of the outer and/or inner membranes where the enzymes of the respiratory chain are functional. In certain preferred embodiments, both the outer and inner mitochondrial membranes are intact.

Mitochondria refers to intact mitochondria from any living species, fungal or animal. Mitochondria are preferably from animals and more preferably from mammals (e.g., rat, mouse, human, rabbit, pig, monkey, ape, etc.). In certain embodiments, the mitochondria may be genetically altered. In other embodiments, the mitochondria may be obtained from at least one cell from a donor, from cell culture of cells from a donor, or from established cells lines. Preferably, the cells are of the same species as the animal to which the test compound is intended to be applied. The cells may be obtained by a biopsy, preferably from the patient or a close relative. Biopsied cells are preferably grown in tissue culture using standard conditions. In the most preferred embodiment, the cells are autologous. In other embodiments, the mitochondria may be obtained from any tissue or type of cell (e.g., hepatocytes, myocytes, fibroblasts, chondrocytes, neurons, osteoblasts, pancreatic islet cells).

Test compound and chemical compound are used interchangeably and refer to any chemical compound such as organic compounds, inorganic compounds, organometallic compounds, and metals. In preferred embodiments of the invention, the compound is a drug or a chemical compound in the stages of drug development. The chemical compound may also be a metabolite of a drug such as a drug metabolized by a P450 enzyme. Drugs include peptide drugs, protein drugs, polynucleotides, oligonucleotides, antibiotics, anti-viral agents, steroidal agents, anti-inflammatory agents, anti-neoplastic agents, antigens, vaccines, antibodies, decongestants, antihypertensives, sedatives, birth control agents, progestational agents, anti-cholinergics, analgesics, anti-depressants, anti-psychotics, β-adrenergic blocking agents, diuretics, cardiovascular active agents, non-steroidal anti-inflammatory agents, nutritional agents, etc. In another preferred embodiment, the test compound is an environmental toxin. In yet another preferred embodiment, the compound is a naturally occurring substance (e.g., natural product).

Therapeutically effective amount refers to the amount of an agent or drug needed to elicit the desired biological response. For example, in the case of anti-psychotic medication, the therapeutically effective amount would decrease or lessen the severity of psychotic symptoms such as hallucinations and delusions.

Neuroleptic and antipsychotics are used interchangeably and refer to drugs typically used to treat psychosis and schizophrenia. These terms encompass both typical and atypical (novel) antipsychotics. Examples include, but are not limited to, haloperidol, quetiapine, risperidone, olanzapine, promazine, chlorpromazine, trazodone, clozapine, thioridazine, molindone, prochlorperazine, moperone, trifluperazine, thiothixene, droperidol, fluphenazine, pimozide, trifluperidol, benperidol, spiroperidol, chlorprothixene, methotrimeprazine, mesoridazine, loxapine, pericyazine, piperacetazine, fluspirilene, pipotiazine, flupenthixol decanoate, and perphenazine. Also, included in this definition are metabolites of these drugs.

### EXAMPLES

### Example 1-Polarographic Assay of Respiration-Dependent Functions

Liver mitochondria are isolated from Sprague Dawley CD rats by differential centrifugation essentially as described previously (Modica-Napolitano et al., “Mitochondrial toxicity of cationic photosensitizers for photochemotherapy” Cancer Res. 50:7876-7881, 1990; incorporated herein by reference). Mitochondrial respiration is measured polarographically using a Clark oxygen electrode. To test for drug effect, an appropriate concentration of drug is introduced at the beginning of the assay, prior to the addition of substrate. The test substance (e.g., drug) is thus present continuously in the assays as the basal, coupled and uncoupled rates are successively determined. The solvent in which the drug is dissolved is tested alone as the control.

Stimulation of basal respiration by the drug indicates uncoupling. If inhibition of the coupled rate is observed, this could be due to inhibition of ATP synthetase function, or to inhibition of electron transport. If the addition of uncoupler fails to relieve the inhibition, then the drug is inhibiting electron transport; and if the addition of uncoupler relieves the inhibition, then the drug is inhibiting ATP synthetase function.

Information about site specific electron transport inhibition can be obtained with this assay by using substrates that donate electrons to the respiratory chain at different branch sites. For example, by separately including either succinate which donates to complex II or glutamate+malate which donates electrons to complex I, or other organic acids which donate through electron-transferring flavoprotein (ETF).

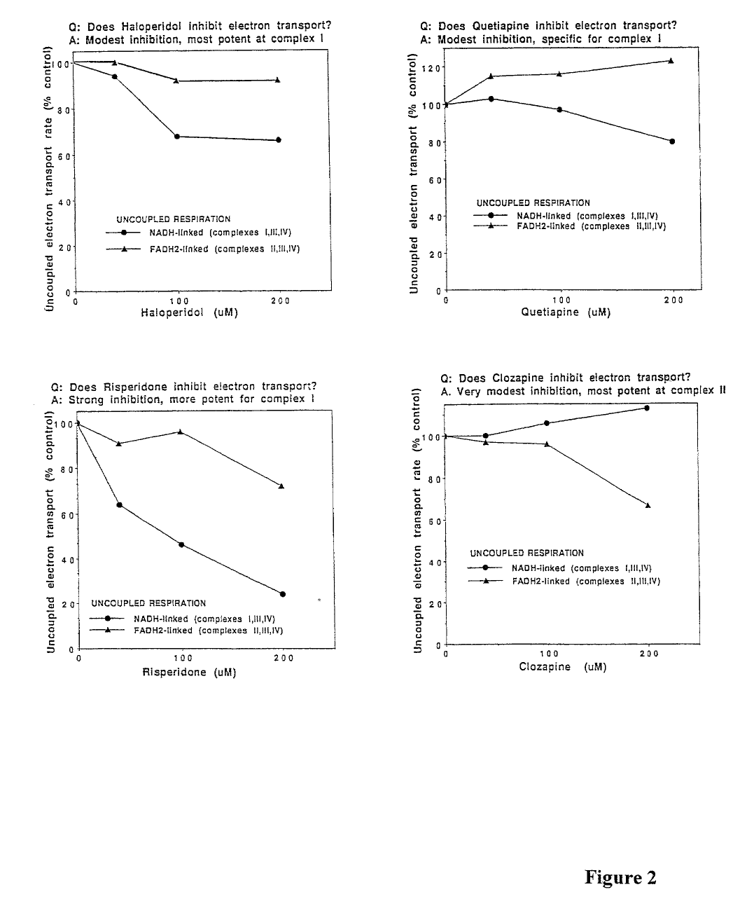

Preliminary data for haloperidol, quetiapine, risperidone, and clozapine in which 2,4-dinitrophenol was added to elicit maximal electron transport rates are shown in FIG. 2. Rates were measured as the rate of oxygen consumption. A slower rate in the presence of the drug indicates inhibition somewhere in the electron transport chain. The tests were performed with different substrates that donate electrons at either respiratory complex I (glutamate+malate) or at complex II (succinate) to gain information about site-specific inhibition. Chlorpromazine and haloperidol inhibited electron transport specifically at respiratory complex I. Quetiapine had a slight effect and was also specific for complex I. Thioridazine and risperidone inhibited electron transport markedly but not specifically at either complex I or II. Clozapine was the only drug that specifically inhibited at respiratory complex II. These results are summarized in Table 1 below.

TABLE 1
Summary of drug effects on mitochondrial bioenergetic function.

| | El. Transport | El. Transport | ATP | |
|---|---|---|---|---|
| Compound | Site I | Site II | Synthesis | Uncoupling |
| Chlorpromazine | +++ | + | − | ++ |
| Thioridazine | +++ | + | − | ++ |
| Haloperidol | ++ | − | − | − |
| Clozapine | − | ++ | + | sl |
| Risperidone | +++ | + | − | ++ |
| Quetiapine | + | − | − | + |

Qualitative comparison of effects: −, no effect; sl, slight effect; number of +’s is proportional to the magnitude of the inhibitory effect. Effects reported here were at 500 μM drug.

Chlorpromazine, thioridazine, haloperidol, and risperidone proved to be potent inhibitors of electron transport; all of these drugs are associated with tardive dyskinesia. Quetiapine and clozapine showed very mild effects. Neither of these drugs, quetiapine or clozapine, are as yet associated with a significant incidence of tardive dyskinesia.

Normally electron transport proceeds at a slow basal rate in the presence of an oxidizable substrate unless the H⁺ gradient is dissipated by a need for ATP synthesis. However, if the H⁺ gradient is dissipated by the addition of a drug that alters membrane permeability to H⁺ , respiration will be stimulated unproductively without concomitant ATP synthesis so that energy is wasted. Haloperidol, quetiapine, risperidone, and clozapine were added to mitochondria respiring in the basal state (FIG. 3). If respiration (i e., oxygen consumption) is stimulated, uncoupling is inferred. A known uncoupler such as 2,3-dinitrophenol stimulates basal respiration 6-10 fold. All of the drugs except haloperidol had some uncoupling action, but the effects were very mild. Chlorpromazine, thoridazine, risperidone, and quetiapine were more potent than clozapine (see Table 1 supra).

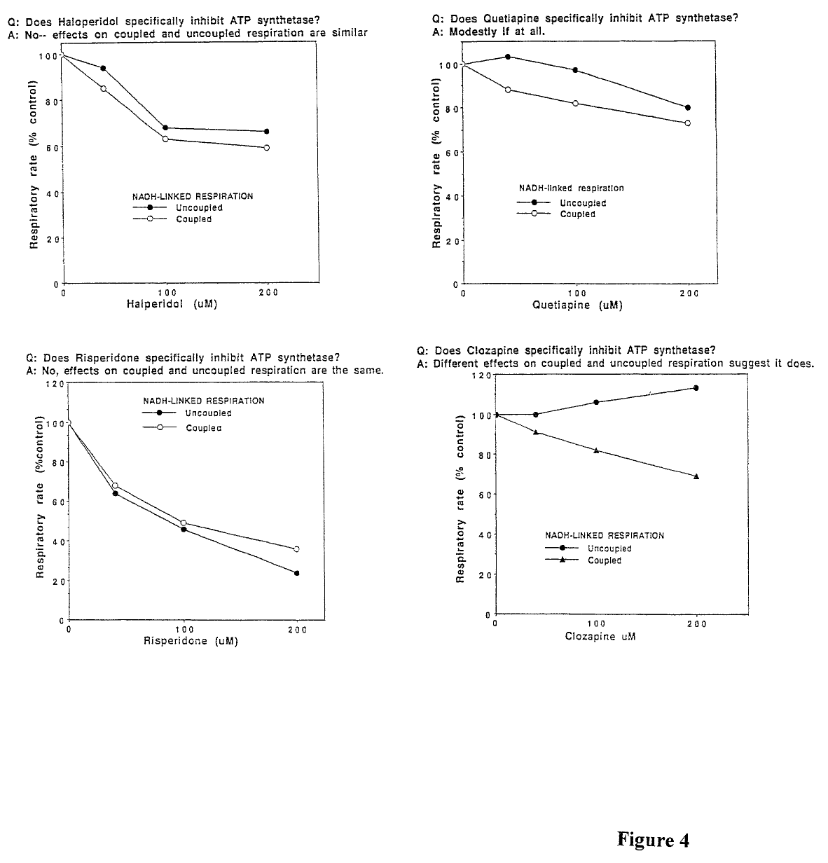

When coupling is intact, basal respiration is stimulated by the addition of ADP which stimulates ATP synthesis, as electron transport is stepped up to re-establish the H⁺ gradient that is used by the ATP synthetase enzyme. If the drug inhibits this coupled respiration rate, the inhibition could be due either to inhibition of the ATP synthetase itself or to inhibition of the electron transport rate that supports the ATP synthesis. Haloperidol, quetiapine, risperidone, and clozapine were tested for inhibition of maximal electron transport rates in the presence and absence of a known uncoupler (FIG. 4). Inhibition of the coupled, but not the uncoupled respiration rate pinpoints the inhibition to the ATP synthetase or to one of the supporting transport processes such as phosphate or ADP/ATP transporters.

Clozapine was the only one of the five drugs tested that appeared to inhibit ATP synthetase (see Table 1 supra). Since the polarographic screening assay is indirect this result should be confirmed by direct assay of ATP synthetase enzyme activity; if direct inhibition is not observed, likely candidates for inhibition are ADP/ATP translocase and phosphate transporters which can be assayed separately.

### Example 2-Enzyme Assays for Specific Respiratory Complexes

The following standard assays for enzymes involved in mitochondrial respiration have been previously reported (Ara et al., “Mechanisms of mitochondrial photosensitization by the cationic dye, N,N-Bis(2-ethyl-1,3-dioxylene)kryptocyanine (EDKC): preferential inactivation of complex I in the electron transport chain” Cancer Res. 47:6580-6585, 1987; Modica-Napolitano et al, “Mitochondrial toxicity of cationic photosensitizers for photochemotherapy” Cancer Res. 50:7876-7881, 1990; each of which is incorporated herein by reference).

Succinate cytochrome c reductase activity (complexes II and III) is determined spectrophotometrically by monitoring the increase in absorbance over time due to the reduction of added cytochrome c when succinate is added.

Rotenone-sensitive NADH-cytochrome c reductase (complexes I and III) is measured by monitoring the reduction of added cytochrome c upon addition of NADH.

Cytochrome c oxidase activity (complex IV) is determined spectrophotometrically by monitoring the oxidation of reduced cytochrome c.

Succinate dehydrogenase activity (complex II) is measured spectrophotometrically at 600 nm, by monitoring the reduction of the artificial electron acceptor, DICP.

Reactions of ATP synthetase. The forward reaction (ATP synthesis) is inferred from oligomycin-sensitive Pᵢ disappearance in the presence of ADP (Modica-Napolitano et al., “Mitochondrial toxicity of cationic photosensitizers for photochemotherapy” Cancer Res. 50:7876-7881, 1990; incorporated herein by reference). Alternatively, F₀F₁ ATPase (reverse reaction) is measured spectrophotometrically at 340 nm by coupling an ATP-regenerating reaction (pyruvate kinase) to the oxidation of NADH via lactate dehydrogenase essentially as described previously (Modica-Napolitano et al., “Basis for the selective cytotoxicity of Rhodamine 123” Cancer Res. 47:4361-4365, 1987; incorporated herein by reference). ATP is added to start the reaction, and the oligomycin-sensitive rate is defined as the rate after adding ATP minus the rate after adding 10 μg oligomycin.

Reduced minus oxidized cytochrome difference spectra. Crossover points in reduced-oxidized difference spectra (Birch-Machin et al, “Identification of mitochondrial dysfunction at couple site II” Chapter 27 207-218, 1993; incorporated herein by reference) can determine the site of electron transport inhibition of a drug. The reduced fraction of cytochromes aa₃, b, cc₁, in mitochondria is determined by wavelength scanning using an Aminco-DW2a spectrophotometer.

Mitochondrial swelling assay. Mitochondria are incubated in respiratory medium, and the drug is added to one of a sample-reference cuvette pair. If swelling is induced, the absorbance of the test cuvette decreases relative to the cuvette. The absorbance is monitored at 540 nm over time to measure the rate of swelling compared to a control assay of vehicle only.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A method of determining whether a chemical compound affects bioenergetic function in isolated mitochondria, the method comprising the steps of:
providing a chemical compound;
providing at least one intact mitochondrion;
contacting the chemical compound with mitochondrion; and
detecting a change in a characteristic as a result of contacting the chemical compound with mitochondrion.

2. The method of claim 1 wherein the chemical compound is a drug.

3. The method of claim 1 wherein the chemical compound is a neuroleptic.

4. The method of claim 1 wherein the chemical compound is an anti-neoplastic drug.

5. The method of claim 1 wherein the chemical compound is a psychiatric medication.

6. The method of claim 1 wherein the chemical compound is a metabolized form of a drug.

7. The method of claim 1 wherein the mitochondrion is in vitro.

8. The method of claim 1 wherein the mitochondrion is in vivo.

9. The method of claim 1 wherein the mitochondrion are from an animal source.

10. The method of claim 1 wherein the mitochondrion are from a mammalian source.

11. The method of claim 1 wherein the mitochondrion are from a rodent source.

12. The method of claim 1 wherein the mitochondrion are from a rat.

13. The method of claim 1 wherein the mitochondrion are from a human.

14. The method of claim 1 wherein the mitochondrion are from an animal to be treated.

15. The method of claim 1 wherein the mitochondrion are from a fungus.

16. The method of claim 1 wherein the mitochondrion are from yeast.

17. The method of claim 1 wherein the characteristic is oxygen concentration.

18. The method of claim 1 wherein the characteristic is a spectral characteristic of a protein in the mitochondrial respiratory chain.

19. The method of claim 1 wherein the characteristic is ATP concentration.

20. The method of claim 1 wherein the characteristic is ADP concentration.

21. The method of claim 1 wherein the characteristic is a concentration of an electron donor.

22. The method of claim 1 wherein the characteristic is FADH₂ concentration.

23. The method of claim 1 wherein the characteristic is NADH concentration.

24. A system comprising at least one intact mitochondrion, and a means of measuring a change when the mitochondrion is contacted with a chemical compound.

25. The system of claim 24 wherein the system further comprises a chemical compound.

26. The system of claim 25 wherein the chemical compound is a drug.

27. The system of claim 25 wherein the chemical compound is a neuroleptic.

28. The system of claim 25 wherein the chemical compound is an anti-neoplastic drug.

29. The system of claim 25 wherein the chemical compound is a psychiatric medication.

30. The system of claim 25 wherein the chemical compound is a metabolized form of a drug.

31. The system of claim 24 wherein the mitochondrion is from an animal source.

32. The system of claim 24 wherein the mitochondrion is from a mammalian source.

33. The system of claim 24 wherein the mitochondrion is from a rodent source.

34. The system of claim 24 wherein the mitochondrion is from a rat.

35. The system of claim 24 wherein the mitochondrion is from a human.

36. The system of claim 24 wherein the mitochondrion is from an animal to be treated.

37. The system of claim 24 wherein the change is a change in oxygen concentration.

38. The system of claim 24 wherein the change is a change in spectral characteristic of a protein in the mitochondrial respiratory change.

39. The system of claim 24 wherein the change is a change in ATP concentration.

40. The system of claim 24 wherein the change is a change in ADP concentration.

41. The system of claim 24 wherein the change is a change in concentration of an electron donor.

42. The system of claim 24 wherein the change is a change in FADH₂ concentration.

43. The system of claim 24 wherein the change is a change in NADH concentration.

44. The system of claim 24 wherein the means is an oxygen electrode.

45. The system of claim 24 wherein the means is a spectrophotometer.

46. The system of claim 24 wherein the means is a standard assay of measuring a concentration of a chemical compound selected from the group consisting of ATP, ADP, FADH₂, NADH, and O₂.

47. The method of claim 1, the method comprises the additional step of:
assaying for a specific respiratory complex.

48. The method of claim 47 wherein the specific respiratory complex is succinate cytochrome c reductase.

49. The method of claim 47 wherein the specific respiratory complex is rotenone-sensitive NADH-cytochrome c reductase.

50. The method of claim 47 wherein the specific respiratory complex is cytochrome c oxidase.

51. The method of claim 47 wherein the specific respiratory complex is succinate dehydrogenase.

52. The method of claim 47 wherein the specific respiratory complex is ATP synthetase.

53. A method of predicting the clinical side effects of a chemical compounds, the method comprising the steps of:
providing a chemical compound;
providing at least one mitochondrion;
contacting the chemical compound with mitochondrion;
measuring at least one characteristic of mitochondrial respiration; and
comparing results of measurement to the measurements of known drug.

54. A pharmaceutical composition comprising
a therapeutically effective amount of a drug; and
an agent known to bypass or ameliorate the bioenergetic lesion caused by the drug.

55. The pharmaceutical composition of claim 54 wherein the agent is a vitamin.

56. The pharmaceutical composition of claim 54 wherein the vitamin is coenzyme Q.

57. The pharmaceutical composition of claim 54 wherein the agent is hydroxybutyrate.

58. The pharmaceutical composition of claim 54 wherein the agent is an alternative respiratory substrate.
